(19) United States
(12) Patent Application Publication
(10) Pub. No.: US 2025/0380864 A1
(43) Pub. Date: Dec. 18, 2025

(54) INTRAVASCULAR ENDOSCOPY BALLOON

(71) Applicant: Northwestern University, Evanston, IL (US)

(72) Inventors: Ahsun RIAZ, Evanston, IL (US); Ali Husnain, Evanston, IL (US)

(21) Appl. No.: 19/238,213

(22) Filed: Jun. 13, 2025

Related U.S. Application Data
(60) Provisional application No. 63/660,885, filed on Jun. 17, 2024.

(51) Int. Cl. A61B 1/313; A61B 1/00; A61B 1/018; A61B 1/04; A61B 1/05; A61B 1/06

(52) U.S. Cl. CPC A61B 1/3137; A61B 1/00082; A61B 1/00119; A61B 1/00147; A61B 1/018; A61B 1/046; A61B 1/05; A61B 1/0676

(57) ABSTRACT

Provided herein is an intravascular endoscope with an improved balloon design. The endoscope comprises an anterior balloon with a camera, light source, and working channel port for visualization and therapeutic access. One or more posterior balloons are positioned inferior to, at the same level as, or superior to the anterior balloon. Inflation of the one or more posterior balloons pushes the anterior balloon against the blood vessel wall while maintaining a channel for blood flow past the anterior balloon. The endoscope enables real-time, direct visualization of the interior of blood vessels for diagnosis and treatment of vascular diseases while preserving critical blood flow. Additional imaging modalities, such as ultraviolet and infrared, can be incorporated to enhance tissue characterization. Visual markers on devices inserted through the working channel improve visibility and guidance under endoscopic imaging. The endoscope is compatible with arteries and veins throughout the body and provides advantages over traditional imaging techniques for managing vascular disease.

### CROSS REFERENCE TO RELATED APPLICATION

The present application claims priority to U.S. Provisional Patent Application No. 63/660,885, filed Jun. 17, 2024, which is incorporated herein by reference in its entirety.

### FIELD

Provided herein are endoscopes with a pair of balloons mounted thereon that find use in angioscopy and cardioscopy. In particular, the endoscope-mounted balloons are inflated with transparent fluid and apposed to the walls of a blood vessel or the heart, stably positioning the endoscope within the treatment site for visualization of and access to the adjacent structures through the balloon while allowing blood to pass by the endoscope and balloons through the vessel.

### BACKGROUND

Angioscopy and cardioscopy are performed as a part of highly invasive surgical procedures that require cardiac bypass to allow for segments of the heart and/or blood vessels (arteries and veins) to be void of blood to allow visualization of the walls. However, given the level of invasiveness of such procedures, cameras are rarely used within blood vessels and the heart for more routine and less invasive procedures.

### SUMMARY

Provided herein are endoscopes with a pair of balloons mounted thereon that find use in angioscopy and cardioscopy. In particular, the endoscope-mounted balloons are inflated with transparent fluid and apposed to the walls of a blood vessel or the heart, stably positioning the endoscope within the treatment site for visualization of and access to the adjacent structures through the balloon while allowing blood to pass by the endoscope and balloons through the vessel.

In some embodiments, provided herein are endoscopes comprising an elongated body having a proximal end and a distal end; an anterior balloon mounted on the distal end of the endoscope, wherein the anterior balloon comprises (or encompasses) a camera, a light source, and a working channel port; posterior balloon(s) mounted on the distal end of the endoscope and positioned inferior to, at the same level as, or superior to the anterior balloon; wherein inflation of the posterior balloon(s) pushes the anterior balloon against the wall of a blood vessel while allowing blood flow past the anterior balloon.

In some embodiments, the anterior balloon and posterior balloon(s) are separately inflatable.

In some embodiments, the anterior balloon and posterior balloon(s) are in fluid communication and inflation of the balloons is controlled by a single inflation lumen.

In some embodiments, the posterior balloon is shaped to leave a blood flow channel when inflated.

In some embodiments, the camera and light source are positioned to enable imaging of the blood vessel wall through the anterior balloon when the anterior balloon is pressed against the vessel wall.

In some embodiments, the working channel port allows passage of instruments from the proximal end of the endoscope to the blood vessel wall.

In some embodiments, the endoscope further comprises an ultraviolet light source and ultraviolet camera.

In some embodiments, the endoscope further comprises an infrared light source and infrared camera.

In some embodiments, provided herein, are systems comprising endoscopes and one or more intravascular devices configured to be inserted through the working channel port, wherein the intravascular devices comprise visual markers to enhance visibility on the camera.

In some embodiments, provided herein are methods of visualizing the interior of a blood vessel, the method comprising advancing an endoscope described herein into the blood vessel; positioning the distal end of the endoscope near a region of interest in the blood vessel; inflating the posterior balloon(s) to push the anterior balloon against the blood vessel wall; and imaging the blood vessel wall using the camera and light source on the anterior balloon.

In some embodiments, the method further comprises passing an instrument through the working channel port to interact with the blood vessel wall. In some embodiments, the instrument comprises a flexible needle that can be advanced through the working channel, a wire with visual and fluoroscopic markers, and/or flexible biopsy forceps.

In some embodiments, the method further comprises capturing an image of the blood vessel wall in ultraviolet or infrared wavelengths.

In some embodiments, the region of interest comprises a vascular lesion, stenosis, plaque, thrombus, or aneurysm.

In some embodiments, the camera, working channel, and light source of the endoscope is forward-facing. In some embodiments, the balloon has a compliant aspect with the working channel going through it. In some embodiments, the balloon also has a component adjacent to the endoscope that is non-compliant. In some embodiments, the combination of compliant and non-compliant components allows the balloon to deflate in a way that facilitates re-capturing the intra-vascular endoscope in the sheath (FIG. 3).

### BRIEF DESCRIPTION OF THE DRAWINGS

FIG. 1A depicts an endoscope with a balloon in accordance with the present disclosure including, (1) wire going through the endoscope, (2) clear balloon filled with clear fluid (anterior), (3) balloon (posterior), (4) camera, (5) working channel opening, (6) channel to inflate/deflate the balloons, and (7) blood vessel wall.

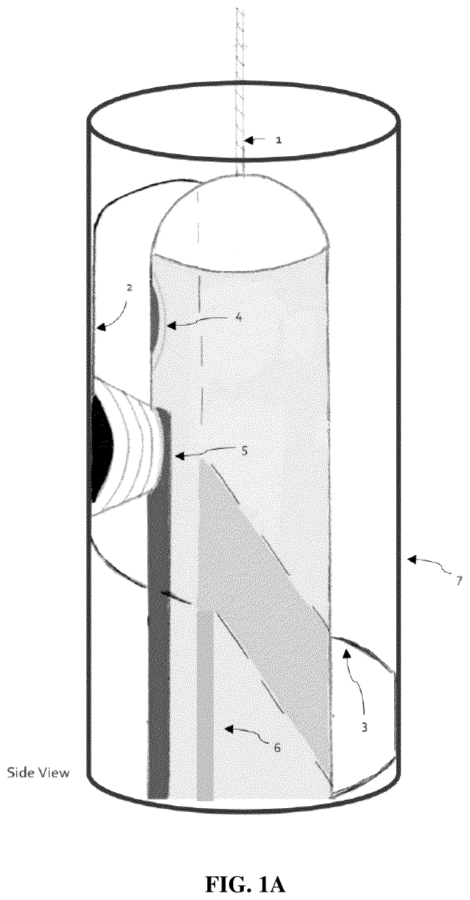

FIG. 1B depicts the endoscope with a balloon of FIG. 1A including, (1) wire going through the endoscope, (2) clear balloon filled with clear fluid (anterior), (3) balloon (posterior), (4) camera, (5) working channel opening, (6) channel to inflate/deflate the balloons, and (7) blood vessel wall, now with the flow of blood around the endoscope shown in red.

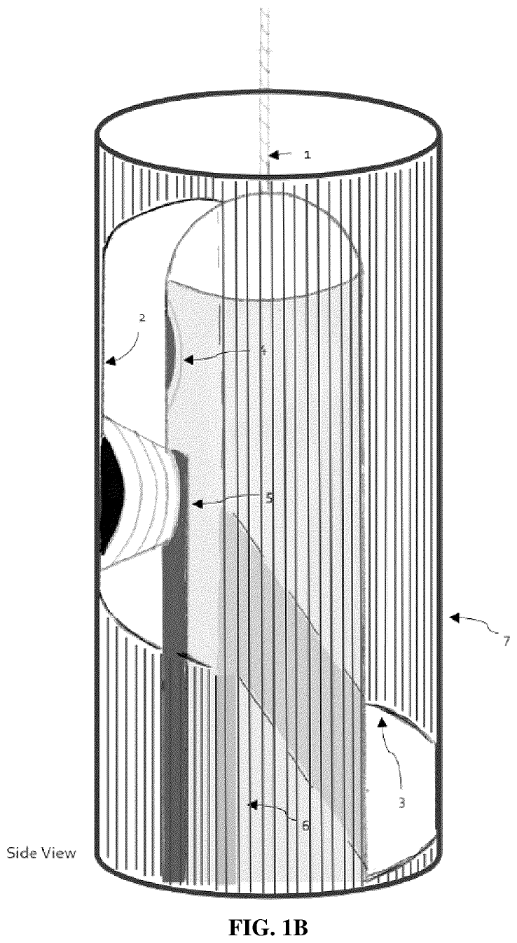

FIG. 2A depicts a top view of an endoscope with a balloon in accordance with the present disclosure including, (1) wire going through the endoscope, (2) clear balloon filled with clear fluid (anterior), (3) balloon (posterior), (4) camera, and (5) blood vessel wall.

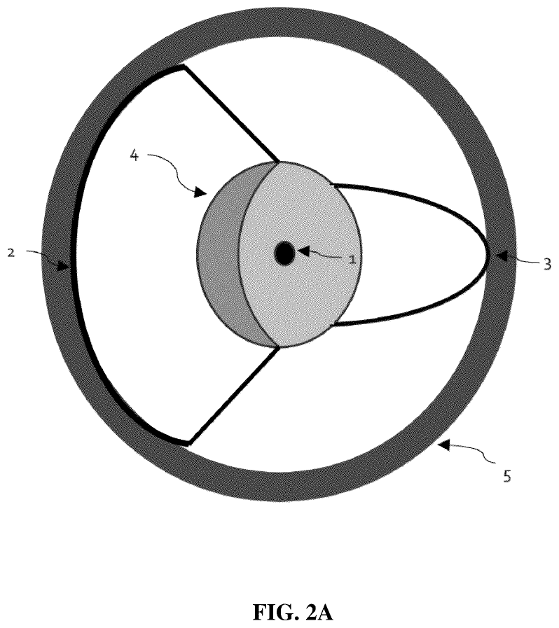

FIG. 2B depicts a top view of FIG. 2A, including, (1) wire going through the endoscope, (2) clear balloon filled with clear fluid (anterior), (3) balloon (posterior), (4) camera, and (5) blood vessel wall, now with the flow of blood around the endoscope shown in red.

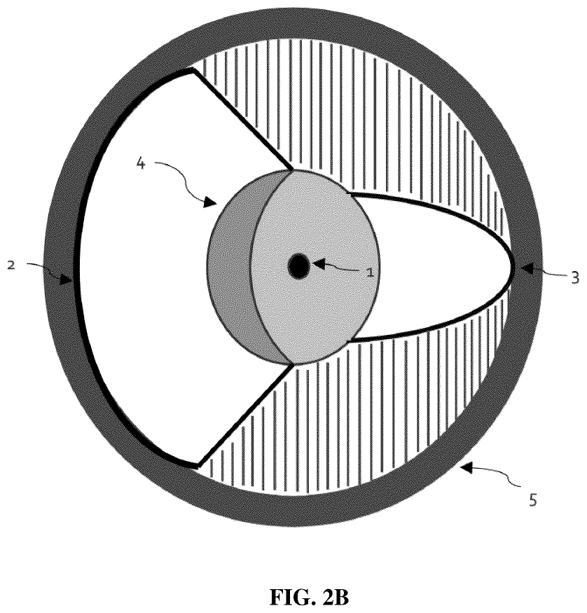

FIG. 3 depicts an exemplary forward-facing intravascular endoscope. This includes: (1) intravascular endoscope; (2) working channel (for wire advancement); (3) forward-facing camera; (4) forward-facing light source; (5) clear balloon filled with clear fluid; (6) reinforced part of the balloon to maintain shape to help with re-sheathing once balloon is deflated; and (7) blood vessel wall with atherosclerosis.

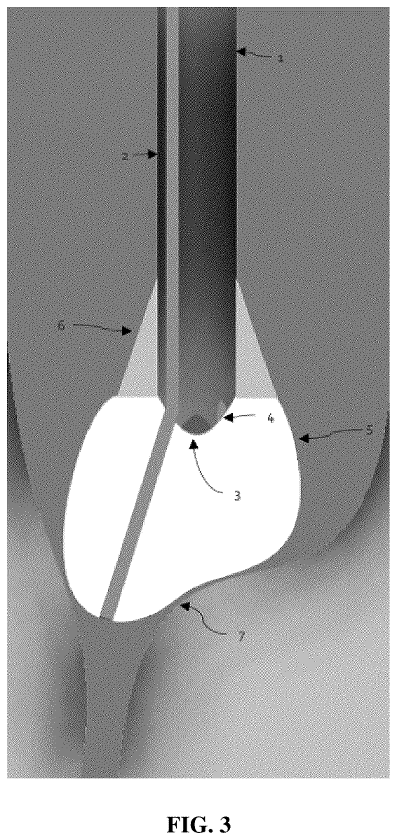

### DETAILED DESCRIPTION

Provided herein are endoscopes with a pair of balloons mounted thereon that find use in angioscopy and cardioscopy. In particular, the endoscope-mounted balloons are inflated with transparent fluid and apposed to the walls of a blood vessel or the heart, stably positioning the endoscope within the treatment site for visualization of and access to the adjacent structures through the balloon while allowing blood to pass by the endoscope and balloons through the vessel.

The endoscopes provided herein may comprise any features, components, materials, etc. understood in the field of endoscopy and intravascular imaging. The scopes may have components and designs similar to commercially available endoscopes, with the addition of the specially designed balloon system.

In some embodiments, the endoscope comprises one or more working channels or lumens extending from the proximal to the distal end to allow insertion of instruments, irrigation, and/or aspiration. A dedicated inflation lumen may be included to independently inflate the anterior and posterior balloons. Alternatively, the anterior and posterior balloons may share an inflation lumen, with the posterior balloon shaped to leave a blood flow channel when inflated. In some embodiments, the channels allow materials (e.g., clear fluid to fill the balloon) to be passed through the endoscope (e.g., from the control end to the distal end) and provide connections between the controls and the working components at the distal end of the endoscope (e.g., camera, light source, orthogonal working channel, wires, etc.).

The anterior balloon is positioned on the distal end of the endoscope and comprises a camera, a light source, and a working channel exit port. In some embodiments, the camera and light source are positioned on the anterior surface of the balloon to enable imaging of the vessel wall when the balloon is inflated. The working channel port allows instruments to reach the vessel wall. In some embodiments, the anterior balloon is 1-6 cm in length and 0.5-2 cm in diameter when inflated, though other sizes may be appropriate depending on the target vessel. In some embodiments, the anterior balloon is configured to, upon inflation, extend from the periphery of the endoscope to the wall of the working site (blood vessel). Upon inflation, the anterior balloon encompasses 20%-60% (e.g., 20%, 25%, 30%, 35%, 40%, 45%, 50%, 55%, 60%, or ranges therebetween) of the cross-sectional area of the blood vessel.

The posterior balloon(s) is/are positioned proximal to, at the same level as, or distal to the anterior balloon on the endoscope shaft. Its purpose is to push the anterior balloon against the vessel wall for imaging while maintaining a blood flow channel in the vessel lumen. In some embodiments, the posterior balloon(s) is/are 1-6 cm in length and 0.5-2 cm in diameter when inflated, though other sizes may be appropriate depending on the target vessel and positioning relative to the anterior balloon. The posterior balloon(s) may be shaped as incomplete circles, helixes, or other geometry that leaves a channel for blood flow when inflated. In some embodiments, the posterior balloon(s) is/are configured to, upon inflation, extend from the periphery of the endoscope to a wall of the blood vessel opposite the working site. Upon inflation, the posterior balloon(s) encompass 5%-30% (e.g., 5%, 10%, 15%, 20%, 25%, 30%, or ranges therebetween) of the cross-sectional area of the blood vessel.

In some embodiments, the anterior and posterior balloons are inflated by the same lumen and inflate simultaneously. The posterior balloon's shape ensures a blood flow channel is maintained. In other embodiments, the anterior and posterior balloons have separate inflation lumens, allowing the posterior balloon to be inflated first to position the anterior balloon, followed by inflation of the anterior balloon for imaging and access. In some embodiments, when both balloons are inflated within a treatment site, the balloons occlude 25%-75% (e.g., 25%, 30%, 35%, 40%, 45%, 50%, 55%, 60%, 65%, 70%, 75%, or ranges therebetween) of the cross-sectional area of the blood vessel. In some embodiments, when both balloons are inflated 25%-75% (e.g., 25%, 30%, 35%, 40%, 45%, 50%, 55%, 60%, 65%, 70%, 75%, or ranges therebetween) of the cross-sectional area of the blood vessel remains unoccluded.

The balloons may be made of compliant or semi-compliant materials such as polyurethane, silicone elastomer, or other transparent, biocompatible materials. In some embodiments, the balloons are attached to the endoscope shaft by heat bonding, adhesives, or other methods known in the art. In some embodiments, the anterior balloon is transparent (e.g., at least 70% transparent (e.g., >70%, >75%, >80%, >90%, >95%)), for example, to the wavelengths of light emitted by the light source. The posterior balloon(s) may be transparent or opaque.

In some embodiments, the balloons are filled with any suitable clear fluid. In some embodiments, the fluid is a gas, such as air, CO₂, N₂, etc. In some embodiments, the clear fluid is a liquid, such as water or saline.

In some embodiments, the balloon material can be any of polyesters, polyamides, nylon 12, nylon 11, polyamide 12, block copolymers of polyether and polyamide, PEBAX, polyurethanes, block copolymers of polyether and polyester, or any other materials suitable for construction of compliant biocompatible balloons, such as those used for angioplasty or other balloon procedures. The materials selected for the balloon result in a transparent balloon, capable of allowing light to pass through the balloon (e.g., from the endoscope light source) and for the camera to view the treatment/diagnostic site through the balloon.

In some embodiments, fluid is passed through the length of the endoscope and to the balloon via balloon channel. In some embodiments, fluid source is located at the proximal end of the endoscope. In some embodiments, a pressure gauge measures and/or regulates the pressure within the balloon.

In some embodiments, endoscopes herein comprise a light source and/or illumination subsystem within the distal tip that is capable of illuminating the treatment/diagnostic site (e.g., through the anterior balloon). In some embodiments, the light source is an LED, fiber optic light emitter, or other type of light. In some embodiments, a light source and/or illumination subsystem comprises one or more of light emitter, light diffuser, mirror, window, lens, etc. In some embodiments, the light source is positioned on the working side of the endoscope and configured to direct light orthogonal to the primary axis of the endoscope (e.g., through the anterior balloon). In some embodiments, the light source is positioned and oriented to illuminate the treatment/diagnostic site and/or the distal end of the side working channel.

In some embodiments, endoscopes herein comprise a camera and/or optical subsystem within the distal tip that is capable of obtaining images and/or video of the treatment/diagnostic site (e.g., through the anterior balloon). In some embodiments, the camera and/or optical subsystem comprises a CCD (charge-coupled device) sensor, CMOS (complementary metal-oxide-semiconductor) sensor, sCMOS (scientific CMOS) sensor, EMCCD (electron multiplying CCD), hybrid sensor (e.g., CMOS-CCD or SPAD Arrays), etc. The optical subsystem may comprise components for wired and/or wireless delivery of images and/or video to a user. In some embodiments, images and/or video are delivered to a used in real time. Systems herein may comprise a monitor, screen, or other device that is external to a subject and allows for viewing of images and/or video sent from the optical subsystem. In some embodiments, the camera and/or optical subsystem is positioned on the working side of the endoscope and configured to obtain images/videa orthogonal to the primary axis of the endoscope (e.g., through the anterior balloon). In some embodiments, the camera and/or optical subsystem is positioned and oriented to obtain images/videa of the treatment/diagnostic site and/or the distal end of the side working channel.

In some embodiments, in addition to the visible spectrum camera and light source, some embodiments of the endoscope include illumination and/or optical subsystems capable of (e.g., having components (e.g., sensors, etc.)) ultraviolet and/or infrared imaging functionalities. The incorporation of additional wavelengths may enhance visualization and tissue characterization compared to visible light alone. Instruments inserted through the endoscope working channel, such as biopsy forceps, needles, snares, etc. may be designed with visual markers such as colors, patterns, or shapes to improve their visibility on the endoscopic camera.

In some embodiments, an endoscope comprises electrical and/or data wiring that runs from one or more components at the distal end of the endoscope (e.g., illumination and/or optical subsystems) to the proximal end of the endoscope (e.g., external to the subject). In some embodiments, an endoscope comprises a electrical and/or data wiring channel. Electrical and/or data wiring may comprise power supply lines, signal/data transmission cables, grounding elements, insulation, etc.

In some embodiments, an endoscope comprises a guidewire channel. The guidewire channel runs the length of the endoscope along the primary axis, from the control end to the distal end, and extends out of the distal tip of the endoscope. A guidewire running through the guidewire channel allows the endoscope to be advanced along the guidewire to the treatment/diagnostic site.

In some embodiments, an endoscope comprises a secondary wire channel. The secondary wire channel runs the length of the endoscope along the primary axis, from the control end to the distal end, and extends out of the working side of the endoscope. In some embodiments, the secondary wire channel terminates at the working channel through the anterior balloon. The secondary wire channel provides a side channel to allow advancement of the wire/device through a working channel (e.g., through the anterior balloon). In some embodiments, a working channel extends out of the distal tip of the endoscope (rather than a side). In such embodiments, a secondary wire may also extend out of the distal tip of the endoscope (rather than a side).

In some embodiments, an endoscope comprises a controller located at the proximal end (control end) of the endoscope. The endoscope extends from the controller to the distal end. In some embodiments, the controller remains outside of the subject with the distal end inserted into the subject and at the treatment/diagnostic site. In some embodiments, the controller comprises components that allow the operator (clinician) to maneuver (e.g., steer, advance, etc.) the endoscope, fill/unfill the balloon(s), introduce materials to the treatment/diagnostic site, take samples, operate the camera, illuminate the treatment/diagnostic site, etc. In some embodiments, the controller is in electronic, mechanical, and/or fluid communication with the distal end of the endoscope (or components located at the distal end of the endoscope) via one or more channels running along the primary axis of the endoscope (e.g., through the endoscope).

In some embodiments, an endoscope herein comprises one or more working channels. In some embodiments, a working channel permits one or more tools (e.g., ablation devices, cannulas, dissectors, electrodes, forceps, graspers, knot pushers, laser fibers, needle holders, suction and irrigation instruments, trocars, and other tools) to be passed through the endoscope to the distal end and to the exterior of the endoscope at the diagnostic/treatment site (e.g., out a side or the distal tip of the endoscope).

In particular, endoscopes herein comprise a working channel extending from a port on the working side (or tip) of the distal end of the endoscope through the anterior balloon. This working channel (e.g., side working channel, distal-tip working channel, etc.) allows for the advancement of wires/devices to the diagnostic/treatment site. In some embodiments, the working channel (e.g., side working channel, distal-tip working channel, etc.) is observable by the camera. In some embodiments, the distal end of the working channel (e.g., side working channel, distal-tip working channel, etc.) is observable by the camera. In some embodiments, the working channel is positioned (e.g., angled) to position a device or other element that has been advanced through the side working channel within view of the camera. In some embodiments, the working channel (e.g., side working channel, distal-tip working channel, etc.) is reinforced with one or more (e.g., 1, 2, 3, 4, 5, 6, 7, 8, 9, 10, or more) circular rings to maintain its shape (e.g., funneled shape) in the compliant anterior balloon from the port on the scope to the edge of the balloon. In some embodiments, the working channel comprises a valve to prevent back-bleeding into the endoscope.

In some embodiments, endoscopes herein comprise an outer sheath. The outer sheath is the protective, biocompatible covering that encloses the internal components. In some embodiments, the outer sheath encases and protects internal components such as optical fibers, image sensors, wiring, and working channels; provides a smooth, lubricious surface to facilitate insertion into bodily passages; maintains structural integrity and flexibility; acts as a barrier to bodily fluids (e.g., blood); ensures electrical insulation and patient safety; and/or supports sterilization and reprocessing protocols.

Endoscopes and components thereof may be constructed of any suitable materials, as would be understood by those in the field, such as metals (e.g., steel, stainless steel, tungsten, titanium, aluminum nickel, copper, gold, platinum, and alloys thereof), glass, insulators (e.g., fluorinated ethylene propylene (FEP), perfluoroalkoxy (PFA), polytetrafluoroethylene (PTFE), etc.), elastomeric or polymeric materials (e.g., silicone, polyamides, polyesters, polyetheretherketones, polyetherurethanes, polyimides, polytetrafluoroethylene, polyurethane epoxies, etc.), copolyester elastomers (e.g., ARNITEL), polyether block amide (e.g., PEBAX), polyether polyester block copolymers (e.g., HYTREL), polyolefin elastomers (e.g., ENGAGE), polyurethane elastomer (e.g., PELLETHANE), styrene block copolymers (e.g., EVOPRENE), styrene-butadiene block copolymers (e.g., STYROFLEX), styrene-ethylene-butylene-styrene block copolymers (e.g., KRATON), and thermoplastic vulcanizates (e.g., SANTOPRENE and GEOLAST), etc.), etc. Embodiments herein are not limited by the materials to construct the endoscopes and components herein.

Methods of using the endoscope to visualize and access blood vessels are also provided. The target vessel, such as an artery or vein, is accessed by a guidewire and the endoscope is advanced over the wire to the region of interest. The posterior balloon(s) is/are inflated to push the anterior balloon against the vessel wall. The anterior balloon is then inflated, which apposes the camera and light source to the vessel wall while instruments may be advanced through the working channel. Direct visualization of the vessel wall enables diagnosis of conditions such as plaques, stenoses, dissections, or thrombi. Therapeutic maneuvers such as biopsy, drug delivery, angioplasty, or stent placement may be performed under endoscopic guidance.

The endoscopes may be used in any blood vessel that can accommodate the dimensions of the device. Non-limiting examples include the coronary arteries, aorta, superior and inferior vena cava, iliac arteries and veins, femoral arteries and veins, carotid arteries, and cerebral arteries and veins. Clinical applications include diagnosing and treating cardiovascular diseases such as atherosclerosis, thrombosis, aneurysms, dissections, and embolisms; guiding endovascular interventions; and evaluating the vascular response to therapies.

In some embodiments, the distal end of an endoscope herein is inserted into an artery or vein of a subject and maneuvered to a diagnostic or treatment site within a blood vessel or a cardiac location of a subject. In some embodiments, the endoscope is inserted through and/or to the diagnostic or treatment site within an artery, such as an artery selected from: the aorta, the common carotid artery, the external carotid artery, the triangles of the neck, the internal carotid artery, the subclavian artery, the axillary artery, the brachial artery, the radial artery, the ulnar artery, the descending aorta, the thoracic aorta, the abdominal aorta, a common iliac artery (e.g., the hypogastric artery, the external iliac artery), the femoral artery, the popliteal artery, the anterior tibial artery, the arteria dorsalis pedis, the posterior tibial artery, etc. In some embodiments, the endoscope is inserted through and/or to the diagnostic or treatment site within a vein, such as a vein selected from: a jugular vein, a hepatic vein, a basilic vein, a saphenous vein, a cephalic vein, a superior vena cava, an inferior vena cava, a pulmonary vein, a subclavian vein, a common iliac vein, an axillary vein, a portal vein, a renal vein, a popliteal vein, a brachiocephalic vein, a posterior tibial vein, a vertebral vein, a median cubital vein, an anterior tibial vein, a retromandibular vein, a femoral vein, a vein from a pterygoid plexus, an azygos vein, a superior mesenteric vein, a posterior auricular vein, a superior sagittal sinus vein, a superior ophthalmic vein, an internal iliac vein, a superficial temporal vein, a coronary sinus, a diploic vein, an anterior jugular vein, a common facial vein, a nasofrontal vein, an occipital vein, an external iliac vein, a deep vein of the thigh, an inferior ophthalmic vein, a vein connecting to an inferior petrosal sinus, an inferior thyroid vein, a splenic vein, a median antebrachial vein, a vein connecting to a superior petrosal sinus, a middle thyroid vein, a deep facial vein, a vein connecting to an inferior sagittal sinus, a frontal vein, etc. In some embodiments, the endoscope is inserted through and/or to the diagnostic or treatment site within a cardiac location, such as: the right atrium, the left atrium, the right ventricle, the left ventricle, etc. Embodiments herein may not be limited by the diagnostic/treatment site, the location of insertion of the device into a subject, or the path of the endoscope from the insertion site to the diagnostic/treatment site.

While the embodiments described herein primarily relate to vascular imaging, the principles of the anterior imaging balloon and posterior blood flow-preserving balloon could be applied to other luminal organs such as the gastrointestinal tract, urinary tract, or respiratory tract. The dimensions and materials of the device may need to be adapted to the specific anatomy and physiologic environment.

The embodiments herein find use in a variety of applications in the fields of medicine and surgery, including but not limited to interventional radiology, interventional neuroradiology, neurosurgery, vascular surgery, interventional cardiology, and cardiac surgery. Exemplary disease processes in which the devices herein may find use include, but are not limited:
Vasculitis: Despite the diagnostic advancements, vasculitis remains difficult to diagnose with certainty. With the help of angioscopy, it is possible to have a closer view of the inner lining of the vessel wall and take biopsy samples, minimizing the risk of puncturing the wall. Some embodiments herein allow the differentiation of different types of vasculitis based on their gross appearance, further supplementing the understanding of these diseases.
Coiling of aneurysms: Intravascular visualization enables the proceduralist to assess the diseased blood vessel's condition and identify any abnormalities, i.e., thrombosis and potential intra/post-coiling complications. It allows the advancement and direct evaluation of coils during the procedures to ensure they are in place and there is complete occlusion of the aneurysmal sac.
Evaluation of previously placed stents: Various complications can arise post-stent placement. The stents can occlude. Radiologic studies have limitations as they do not definitively allow evaluation of the inner lining of stents. The angioscopy devices herein allow a close-up, real-time view of the stents to guide interventionalists to manage the problem.
Dissection: Dissection of arteries (a condition where there is a tear inside the vessel wall with a flap limiting blood flow), especially of the aorta, requires immediate attention, especially when there is a risk of rupture. This is a prime example where the devices herein can be used to make definite diagnoses and to manage dissections through wire access of true/false lumen.
Venous disease: Like arterial diseases, angioscopy has diagnostic and therapeutic roles in venous diseases. The devices herein can be used to diagnose deep vein thromboses, assess venous wall integrity, or guide intervention in treating diseased veins, like thrombectomy.

What is claimed is:

1. An endoscope comprising:
an elongated body having a proximal end and a distal end;
an anterior balloon mounted on the distal end of the endoscope, wherein the anterior balloon comprises a camera, a light source, and a working channel port;
one or more posterior balloons mounted on the distal end of the endoscope; wherein inflation of the one or more posterior balloons pushes the anterior balloon against a wall of a blood vessel while allowing blood flow past the anterior balloon.

2. The endoscope of claim 1, wherein the one or more posterior balloons are positioned at the same level as the anterior balloon.

3. The endoscope of claim 1, wherein the one or more posterior balloons are positioned inferior to the anterior balloon.

4. The endoscope of claim 1, wherein the one or more posterior balloons are positioned superior to the anterior balloon.

5. The endoscope of claim 1, wherein the anterior balloon and one or more posterior balloons are separately inflatable.

6. The endoscope of claim 1, wherein the anterior balloon and one or more posterior balloons are in fluid communication and inflation of the balloons is controlled by a single inflation lumen.

7. The endoscope of claim 6, wherein the one or more posterior balloons are shaped to leave a blood flow channel when inflated.

8. The endoscope of claim 1, wherein the camera and light source are positioned to enable imaging of the blood vessel wall through the anterior balloon when the anterior balloon is pressed against the vessel wall.

9. The endoscope of claim 1, wherein the working channel port allows passage of instruments from the proximal end of the endoscope to the blood vessel wall.

10. The endoscope of claim 1, further comprising an ultraviolet light source and ultraviolet camera.

11. The endoscope of claim 1, further comprising an infrared light source and infrared camera.

12. A system comprising the endoscope of claim 1 and one or more intravascular devices configured to be inserted through the working channel port, wherein the intravascular devices comprise visual markers to enhance visibility on the camera.

13. A method of visualizing the interior of a blood vessel, the method comprising:
advancing the endoscope of claim 1 into the blood vessel;
positioning the distal end of the endoscope near a region of interest in the blood vessel;
inflating the one or more posterior balloons to push the anterior balloon against the blood vessel wall; and
imaging the blood vessel wall using the camera and light source positioned on the anterior balloon.

14. The method of claim 13, further comprising inflating the anterior balloon.

15. The method of claim 13, further comprising passing an instrument through the working channel port to interact with the blood vessel wall.

16. The method of claim 13, further comprising capturing an image of the blood vessel wall in ultraviolet or infrared wavelengths.

17. The method of claim 13, wherein the region of interest comprises a vascular lesion, stenosis, plaque, thrombus, or aneurysm.
